(19) United States
(12) Patent Application Publication
(10) Pub. No.: US 2025/0049919 A1
(43) Pub. Date: Feb. 13, 2025

(54) Prevention of Immunological Rejection Using Mesenchymal Stem Cells and Derivatives Thereof

(71) Applicant: CREATIVE MEDICAL TECHNOLOGIES, INC., Phoenix, AZ (US)

(72) Inventors: Thomas Ichim, San Diego, CA (US); Courtney Bartlett, Niceville, FL (US); Timothy Warbington, Phoenix, AZ (US); Amit Patel, Salt Lake City, UT (US)

(73) Assignee: CREATIVE MEDICAL TECHNOLOGIES, INC., Phoenix, AZ (US)

(21) Appl. No.: 18/785,059

(22) Filed: Jul. 26, 2024

Related U.S. Application Data
(60) Provisional application No. 63/518,424, filed on Aug. 9, 2023.

(51) Int. Cl. A61K 39/00; C12N 5/0775

(52) U.S. Cl. CPC A61K 39/4621; A61K 39/461; C12N 5/0663

(57) ABSTRACT

Compositions of matter and treatment protocols for reducing or completely avoiding the need for immune suppression in the context of allograft or xenograft transplantation. Use of mesenchymal stem cells, manipulated mesenchymal stem cells, iPSC derived mesenchymal stem cells, and products of said mesenchymal stem cells for promoting the process of immunological tolerance. In one embodiment the invention provides the use of third party “universal donor” iPSC gene edited mesenchymal stem cells to promote tolerance in context of a solid organ transplant such as renal, cardiac, hepatic or small intestine. In other embodiments endogenous mesenchymal stem cells in composite tissue allografts are activated and/expanded in order to promote tolerogenesis and reduced need for immune suppression.

### CROSS-REFERENCE TO RELATED APPLICATIONS

The present application claims benefit of U.S. Provisional Patent Application Ser. No. 63/518,424, filed on Aug. 9, 2023, titled PREVENTION OF IMMUNOLOGICAL REJECTION USING MESENCHYMAL STEM CELLS AND DERIVATIVES THEREOF, the contents of which are incorporated herein by reference in its entirety.

### FIELD OF THE INVENTION

The teachings herein relate to compositions and methods of reducing the need for immune suppression of allograft and xenograft transplantation through the use of mesenchymal stem cells.

### BACKGROUND OF THE INVENTION

Organ transplantation is the only therapeutic solution for numerous chronic conditions. Unfortunately, the use of transplantation is limited by development of immunological rejection. The use of immune suppressants such as cyclosporin has resulted in significant enhancement of allograft survival in part through overcoming the problem of acute rejection. Unfortunately, current day immune suppressants still allow for development of chronic rejection, which usually accounts for the majority of organ losses. Additionally, immune suppressants typically used for transplantation are known to possess a variety of toxic effects. The administration of immune suppressants has also been shown to correlated with increased risk of both viral and spontaneously induced neoplasia.

There is a need in the art to develop ways of inducing tolerance to transplanted tissues. Although in some animal models this has been achieved by administration of immature dendritic cells, this process is unreliable and clinical feasibility is limited.

The current invention provides the use of mesenchymal stem cells and various manipulations of mesenchymal stem cells in order to induce antigen specific tolerance and to reduce or completely eliminate the need for immune suppressive drugs.

### SUMMARY OF THE INVENTION

Preferred embodiments include methods of promoting immunological tolerance to a transplanted tissue comprising the steps of: a) identifying immunological factors in a potential transplant recipient; b) administering a mesenchymal stem cell population prior to transplantation of said tissue in a manner to alter recipient immune system; c) assessing recipient immune system modifications induced by said mesenchymal stem cell population; d) performing said transplant; and e) tailoring future administration of said mesenchymal stem cells based on alterations of recipient immune response post-transplant.

Preferred methods include embodiments wherein said transplanted tissue is an organ.

Preferred methods include embodiments wherein said transplanted tissue is one or more cellular populations.

Preferred methods include embodiments wherein said transplanted tissue is a composite graft.

Preferred methods include embodiments wherein said immunological tolerance is graft-specific.

Preferred methods include embodiments wherein said immunological tolerance is a state in which the recipient immune system is unresponsive to graft associated antigens.

Preferred methods include embodiments wherein said immunological tolerance is a state in which the recipient immune system is actively suppressing immune responses against graft associated antigens.

Preferred methods include embodiments wherein said suppression of anti-graft immunity is mediated by tolerogenic dendritic cells.

Preferred methods include embodiments wherein said tolerogenic dendritic cells express PD-L1.

Preferred methods include embodiments wherein said tolerogenic dendritic cells express VISTA.

Preferred methods include embodiments wherein said tolerogenic dendritic cells express TIGIT.

Preferred methods include embodiments wherein said tolerogenic dendritic cells express LAG-3.

Preferred methods include embodiments wherein said tolerogenic dendritic cells express TIM3.

Preferred methods include embodiments wherein said tolerogenic dendritic cells secrete IL-10.

Preferred methods include embodiments wherein said tolerogenic dendritic cells secrete soluble TNF-alpha receptor p55.

Preferred methods include embodiments wherein said tolerogenic dendritic cells secrete soluble TNF-alpha receptor p75.

Preferred methods include embodiments wherein said tolerogenic dendritic cells secrete soluble ICAM-1.

Preferred methods include embodiments wherein said tolerogenic dendritic cells secrete soluble HLA-G.

Preferred methods include embodiments wherein said tolerogenic dendritic cells secrete soluble PD-L1.

Preferred methods include embodiments wherein said tolerogenic dendritic cells secrete interleukin-1 receptor antagonist.

Preferred methods include embodiments wherein said tolerogenic dendritic cells secrete interleukin-13.

Preferred methods include embodiments wherein said tolerogenic dendritic cells secrete interleukin-35.

Preferred methods include embodiments wherein said tolerogenic dendritic cells secrete interleukin-20.

Preferred methods include embodiments wherein said tolerogenic dendritic cells secrete interleukin-22.

Preferred methods include embodiments wherein said tolerogenic dendritic cells secrete interleukin-37.

Preferred methods include embodiments wherein said tolerogenic dendritic cells secrete VEGF.

Preferred methods include embodiments wherein said tolerogenic dendritic cells secrete HGF.

Preferred methods include embodiments wherein said tolerogenic dendritic cells secrete PDGF.

Preferred methods include embodiments wherein said tolerogenic dendritic cells secrete angiopoietin.

Preferred methods include embodiments wherein said tolerogenic dendritic cells secrete progesterone induced blocking factor.

Preferred methods include embodiments wherein said tolerogenic dendritic cells suppress proliferation of activated T cells.

Preferred methods include embodiments wherein said tolerogenic dendritic cells suppress interferon production of activated T cells.

Preferred methods include embodiments wherein said tolerogenic dendritic cells suppress interleukin-2 production of activated T cells.

Preferred methods include embodiments wherein said tolerogenic dendritic cells suppress interleukin-6 production of activated T cells.

Preferred methods include embodiments wherein said tolerogenic dendritic cells suppress interleukin-7 production of activated T cells.

Preferred methods include embodiments wherein said tolerogenic dendritic cells suppress interleukin-12 production of activated T cells.

Preferred methods include embodiments wherein said tolerogenic dendritic cells suppress interleukin-15 production of activated T cells.

Preferred methods include embodiments wherein said tolerogenic dendritic cells suppress interleukin-18 production of activated T cells.

Preferred methods include embodiments wherein said tolerogenic dendritic cells suppress interleukin-21 production of activated T cells.

Preferred methods include embodiments wherein said tolerogenic dendritic cells suppress interleukin-17 production of activated T cells.

Preferred methods include embodiments wherein said tolerogenic dendritic cells suppress interleukin-23 production of activated T cells.

Preferred methods include embodiments wherein said tolerogenic dendritic cells suppress interleukin-27 production of activated T cells.

Preferred methods include embodiments wherein said tolerogenic dendritic cells suppress interleukin-33 production of activated T cells.

Preferred methods include embodiments wherein said tolerogenic dendritic cells suppress granzyme production of activated T cells.

Preferred methods include embodiments wherein said tolerogenic dendritic cells suppress perforin production of activated T cells.

Preferred methods include embodiments wherein said tolerogenic dendritic cells suppress RANK ligand production of activated T cells.

Preferred methods include embodiments wherein said tolerogenic dendritic cells suppress TRAIL production of activated T cells.

Preferred methods include embodiments wherein said tolerogenic dendritic cells suppress TRANCE production of activated T cells.

Preferred methods include embodiments wherein said tolerogenic dendritic cells suppress decoy receptor 1 production of activated T cells.

Preferred methods include embodiments wherein said tolerogenic dendritic cells suppress osteopontin production of activated T cells.

Preferred methods include embodiments wherein said tolerogenic dendritic cells increase interleukin-4 production of activated T cells.

Preferred methods include embodiments wherein said tolerogenic dendritic cells increase interleukin-10 production of activated T cells.

Preferred methods include embodiments wherein said tolerogenic dendritic cells increase interleukin-1 receptor antagonist production of activated T cells.

Preferred methods include embodiments wherein said tolerogenic dendritic cells increase interleukin-13 production of activated T cells.

Preferred methods include embodiments wherein said tolerogenic dendritic cells increase interleukin-20 production of activated T cells.

Preferred methods include embodiments wherein said tolerogenic dendritic cells increase interleukin-22 production of activated T cells.

Preferred methods include embodiments wherein said tolerogenic dendritic cells increase interleukin-25 production of activated T cells.

Preferred methods include embodiments wherein said tolerogenic dendritic cells increase interleukin-35 production of activated T cells.

Preferred methods include embodiments wherein said tolerogenic dendritic cells increase interleukin-38 production of activated T cells.

Preferred methods include embodiments wherein said tolerogenic dendritic cells increase TGF-beta production of activated T cells.

Preferred methods include embodiments wherein said tolerogenic dendritic cells increase notch production of activated T cells.

Preferred methods include embodiments wherein said tolerogenic dendritic cells increase GDF-11 production of activated T cells.

Preferred methods include embodiments wherein said tolerogenic dendritic cells increase GDF-15 production of activated T cells.

Preferred methods include embodiments wherein said tolerogenic dendritic cells increase endoglin production of activated T cells.

Preferred methods include embodiments wherein said tolerogenic dendritic cells increase VEGF production of activated T cells.

Preferred methods include embodiments wherein said tolerogenic dendritic cells increase PDGF-BB production of activated T cells.

Preferred methods include embodiments wherein said tolerogenic dendritic cells increase production of activated T cells.

Preferred methods include embodiments wherein said tolerogenic dendritic cells increase leukemia inhibitory factor production of activated T cells.

Preferred methods include embodiments wherein said tolerogenic dendritic cells increase MMP-3 production of activated T cells.

Preferred methods include embodiments wherein said tolerogenic dendritic cells increase MMP-9 production of activated T cells.

Preferred methods include embodiments wherein said tolerogenic dendritic cells increase MMP-13 production of activated T cells.

Preferred methods include embodiments wherein said tolerogenic dendritic cells increase soluble IL-6 receptor production of activated T cells.

Preferred methods include embodiments wherein said tolerogenic dendritic cells increase arginase production of activated T cells.

Preferred methods include embodiments wherein said tolerogenic dendritic cells increase CD73 production of activated T cell

Preferred methods include embodiments wherein said tolerogenic dendritic cells increase exosome production of activated T cells.

Preferred methods include embodiments wherein said exosomes express tetraspanin.

Preferred methods include embodiments wherein said exosomes express membrane bound TGF-beta.

Preferred methods include embodiments wherein said exosomes express membrane bound IL-10.

Preferred methods include embodiments wherein said exosomes express membrane bound IL-35.

Preferred methods include embodiments wherein said exosomes express membrane bound Fas ligand.

Preferred methods include embodiments wherein said exosomes express membrane bound Death Receptor 1.

Preferred methods include embodiments wherein said exosomes express CD1.

Preferred methods include embodiments wherein said exosomes express CD3.

Preferred methods include embodiments wherein said exosomes express CD4.

Preferred methods include embodiments wherein said exosomes express CD8.

Preferred methods include embodiments wherein said exosomes express CD14.

Preferred methods include embodiments wherein said exosomes express CD56.

Preferred methods include embodiments wherein said exosomes express CD73.

Preferred methods include embodiments wherein said exosomes express CD90.

Preferred methods include embodiments wherein said exosomes express CD105.

Preferred methods include embodiments wherein said exosomes express CD132.

Preferred methods include embodiments wherein said exosomes express CD127.

Preferred methods include embodiments wherein said exosomes express PD-L1.

Preferred methods include embodiments wherein said exosomes express CTLA4.

Preferred methods include embodiments wherein said exosomes express BTLA4.

Preferred methods include embodiments wherein said exosomes express low molecular weight hyaluronic acid on their membranes.

Preferred methods include embodiments wherein said immunological tolerance is associated with generation of graft protective T regulatory cells.

Preferred methods include embodiments wherein said graft protective T regulatory cells are capable of suppressing dendritic cell activation.

Preferred methods include embodiments wherein said dendritic cell activation is dendritic cell maturation.

Preferred methods include embodiments wherein said dendritic cell maturation is augmentation of HLA levels on the surface of said dendritic cells.

Preferred methods include embodiments wherein said recipient immunological factor is donor reactive antibodies.

Preferred methods include embodiments wherein said recipient immunological factor is donor reactive T cells.

Preferred methods include embodiments wherein said donor reactive antibodies are complement fixing.

Preferred methods include embodiments wherein said donor reactive antibodies are of the IgG isotype.

Preferred methods include embodiments wherein said donor reactive antibodies are natural antibodies.

Preferred methods include embodiments wherein said natural antibodies are of the IgM isotype.

Preferred methods include embodiments wherein said donor reactive T cells are derived from the thymus.

Preferred methods include embodiments wherein said donor reactive T cells are derived from the bone marrow.

Preferred methods include embodiments wherein said donor reactive T cells express CD3.

Preferred methods include embodiments wherein said donor reactive T cells express CD4.

Preferred methods include embodiments wherein said donor reactive T cells express CD8.

Preferred methods include embodiments wherein said donor reactive T cells express CD56.

Preferred methods include embodiments wherein said donor reactive T cells express CD57.

Preferred methods include embodiments wherein said donor reactive T cells secrete interferon gamma.

Preferred methods include embodiments wherein said donor reactive T cells secrete TNF-alpha.

Preferred methods include embodiments wherein said donor reactive T cells secrete lymphotoxin.

Preferred methods include embodiments wherein said donor reactive T cells secrete TWEAK.

Preferred methods include embodiments wherein said donor reactive T cells secrete granzyme B.

Preferred methods include embodiments wherein said donor reactive T cells secrete perforin.

Preferred methods include embodiments wherein said donor reactive T cells secrete interleukin-1.

Preferred methods include embodiments wherein said donor reactive T cells secrete interleukin-2.

Preferred methods include embodiments wherein said donor reactive T cells secrete interleukin-6.

Preferred methods include embodiments wherein said donor reactive T cells secrete interleukin-7.

Preferred methods include embodiments wherein said donor reactive T cells secrete interleukin-9.

Preferred methods include embodiments wherein said donor reactive T cells secrete interleukin-12.

Preferred methods include embodiments wherein said donor reactive T cells secrete interleukin-15.

Preferred methods include embodiments wherein said donor reactive T cells secrete interleukin-17.

Preferred methods include embodiments wherein said donor reactive T cells secrete interleukin-18.

Preferred methods include embodiments wherein said donor reactive T cells secrete interleukin-21.

Preferred methods include embodiments wherein said donor reactive T cells secrete interleukin-22.

Preferred methods include embodiments wherein said donor reactive T cells secrete interleukin-23.

Preferred methods include embodiments wherein said donor reactive T cells secrete interleukin-27.

Preferred methods include embodiments wherein said donor reactive T cells secrete interleukin-33.

Preferred methods include embodiments wherein said donor reactive T cells express Fas ligand.

Preferred methods include embodiments wherein said mesenchymal stem cell is a plastic adherent cell.

Preferred methods include embodiments wherein said mesenchymal stem cell possesses markers selected from a group comprising of: a) CD73; b) CD105; and c) c-kit.

Preferred methods include embodiments wherein said mesenchymal stem cell further expresses interleukin-1 receptor.

Preferred methods include embodiments wherein said mesenchymal stem cell further expresses interleukin-6 receptor.

Preferred methods include embodiments wherein said mesenchymal stem cell further expresses interleukin-8 receptor.

Preferred methods include embodiments wherein said mesenchymal stem cell further expresses interleukin-11 receptor.

Preferred methods include embodiments wherein said mesenchymal stem cell further expresses interleukin-13 receptor.

Preferred methods include embodiments wherein said mesenchymal stem cell further expresses interleukin-22 receptor.

Preferred methods include embodiments wherein said mesenchymal stem cell further expresses interferon alpha receptor.

Preferred methods include embodiments wherein said mesenchymal stem cell further expresses interferon beta receptor.

Preferred methods include embodiments wherein said mesenchymal stem cell further expresses interferon gamma receptor.

Preferred methods include embodiments wherein said mesenchymal stem cell further expresses OCT4.

Preferred methods include embodiments wherein said mesenchymal stem cell further expresses vegf receptor.

Preferred methods include embodiments wherein said mesenchymal stem cell further expresses c-maf.

Preferred methods include embodiments wherein said mesenchymal stem cell further expresses EGF receptor.

Preferred methods include embodiments wherein said mesenchymal stem cell further expresses FGF-1 receptor

Preferred methods include embodiments wherein said mesenchymal stem cell further expresses leukemia inhibitory factor receptor.

Preferred methods include embodiments wherein said mesenchymal stem cell possesses ability to suppress a mixed lymphocyte reaction.

Preferred methods include embodiments wherein said suppression of said mixed lymphocyte reaction is inhibition of proliferation.

Preferred methods include embodiments wherein said suppression of said mixed lymphocyte reaction is inhibition of proliferation.

Preferred methods include embodiments wherein said proliferation is proliferation of T cells.

Preferred methods include embodiments wherein said proliferating T cells are responding to one or more alloantigens.

Preferred methods include embodiments wherein said proliferating T cells are CD3 expressing T cells.

Preferred methods include embodiments wherein said proliferating T cells are CD4 expressing T cells.

Preferred methods include embodiments wherein said proliferating T cells are CD8 expressing T cells.

Preferred methods include embodiments wherein said proliferating T cells are Th1 cells.

Preferred methods include embodiments wherein said proliferating T cells are Th9 cells.

Preferred methods include embodiments wherein said proliferating T cells are Th17 cells.

Preferred methods include embodiments wherein said proliferating T cells are cytotoxic T cells.

Preferred methods include embodiments wherein said proliferating T cells are helper T cells.

Preferred methods include embodiments wherein said mesenchymal stem cells are derived from tissue based on expression of CD73 and production of hepatocyte growth factor.

Preferred methods include embodiments wherein said mesenchymal stem cells are derived from tissue based on expression of CD73 and production of fibroblast growth factor 1.

Preferred methods include embodiments wherein said mesenchymal stem cells are derived from tissue based on expression of CD73 and production of fibroblast growth factor 2.

Preferred methods include embodiments wherein said mesenchymal stem cells are derived from tissue based on expression of CD73 and production of TLR3.

Preferred methods include embodiments wherein said mesenchymal stem cells are derived from tissue based on expression of CD73 and production of TLR4.

Preferred methods include embodiments wherein said mesenchymal stem cells are derived from tissue based on expression of CD73 and production of TLR5.

Preferred methods include embodiments wherein said mesenchymal stem cells are derived from tissue based on expression of CD73 and production of TLR9.

Preferred methods include embodiments wherein said mesenchymal stem cells are derived from tissue based on expression of CD73 and production of RIG.

Preferred methods include embodiments wherein said mesenchymal stem cells are derived from tissue based on expression of CD73 and production of MDA5.

Preferred methods include embodiments wherein said mesenchymal stem cells are derived from tissue based on expression of CD73 and production of soluble TNF-alpha receptor.

Preferred methods include embodiments wherein said mesenchymal stem cells are derived from tissue based on expression of CD73 and production of interleukin-1 receptor antagonist.

Preferred methods include embodiments wherein said mesenchymal stem cells are derived from tissue based on expression of CD73 and production of interleukin-10.

Preferred methods include embodiments wherein said mesenchymal stem cells are derived from tissue based on expression of CD73 and production of TGF-beta.

Preferred methods include embodiments wherein said mesenchymal stem cells are derived from tissue based on expression of CD73 and production of interleukin-13.

Preferred methods include embodiments wherein said mesenchymal stem cells are derived from tissue based on expression of CD73 and production of endoglin.

Preferred methods include embodiments wherein said mesenchymal stem cells are derived from tissue based on expression of CD73 and production of endosialin.

Preferred methods include embodiments wherein said mesenchymal stem cells are derived from tissue based on expression of CD73 and production of PDL1.

Preferred methods include embodiments wherein said mesenchymal stem cells are derived from tissue based on expression of CD73 and production of GDF11.

Preferred methods include embodiments wherein said mesenchymal stem cells are derived from tissue based on expression of CD73 and production of PDGF-BB.

Preferred methods include embodiments wherein said mesenchymal stem cells are derived from tissue based on expression of CD73 and production of arginase.

Preferred methods include embodiments wherein said mesenchymal stem cells are derived from tissue based on expression of CD73 and production of indolamine 2,3 dioxygenase.

Preferred methods include embodiments wherein said mesenchymal stem cells are derived from tissue based on expression of CD73 and production of galectin-3.

Preferred methods include embodiments wherein said mesenchymal stem cells are derived from tissue based on expression of CD73 and production of galectin-5.

Preferred methods include embodiments wherein said mesenchymal stem cells are derived from tissue based on expression of CD73 and production of galectin-9.

Preferred methods include embodiments wherein said mesenchymal stem cells are derived from tissue based on expression of CD73 and production of LRP.

Preferred methods include embodiments wherein said mesenchymal stem cells are derived from tissue based on expression of CD73 and production of calreticulin.

Preferred methods include embodiments wherein said mesenchymal stem cells are derived from tissue based on expression of CD73 and production of angiopoietin.

Preferred methods include embodiments wherein said mesenchymal stem cells are derived from tissue based on expression of CD73 and production of nitric oxide synthase.

Preferred methods include embodiments wherein said mesenchymal stem cells are derived from tissue based on expression of CD73 and production of PGE2.

Preferred methods include embodiments wherein said mesenchymal stem cells are derived from tissue based on expression of CD73 and production of plasminogen activator inhibitor.

Preferred methods include embodiments wherein said mesenchymal stem cells are derived from tissue based on expression of CD73 and production of cathepsin S.

Preferred methods include embodiments wherein said mesenchymal stem cells are derived from tissue based on expression of CD73 and production of aldehyde dehydrogenase.

Preferred methods include embodiments wherein said mesenchymal stem cells are derived from tissue based on expression of CD73 and production of multidrug resistance efflux pump.

Preferred methods include embodiments wherein said mesenchymal stem cells are derived from tissue based on expression of CD73, c-kit and interleukin-3 receptor.

Preferred methods include embodiments wherein said mesenchymal stem cells are derived from tissue based on expression of CD73, c-kit and interleukin-6 receptor.

Preferred methods include embodiments wherein said mesenchymal stem cells are derived from tissue based on expression of CD73, c-kit and interleukin-7 receptor.

Preferred methods include embodiments wherein said mesenchymal stem cells are derived from tissue based on expression of CD73, c-kit and interleukin-9 receptor.

Preferred methods include embodiments wherein said mesenchymal stem cells are derived from tissue based on expression of CD73, c-kit and interleukin-12 receptor.

Preferred methods include embodiments wherein said mesenchymal stem cells are derived from tissue based on expression of CD73, c-kit and interleukin-15 receptor.

Preferred methods include embodiments wherein said mesenchymal stem cells are derived from tissue based on expression of CD73, c-kit and interleukin-17 receptor.

Preferred methods include embodiments wherein said mesenchymal stem cells are derived from tissue based on expression of CD73, c-kit and interleukin-18 receptor.

Preferred methods include embodiments wherein said mesenchymal stem cells are derived from peripheral blood.

Preferred methods include embodiments wherein said mesenchymal stem cells are derived from adipose tissue.

Preferred methods include embodiments wherein said mesenchymal stem cells are derived from menstrual blood.

Preferred methods include embodiments wherein said mesenchymal stem cells are derived from lymphatic tissues.

205. Preferred methods include embodiments wherein said mesenchymal stem cells are derived from tonsils.

Preferred methods include embodiments wherein said mesenchymal stem cells are derived from fallopian tubes.

Preferred methods include embodiments wherein said mesenchymal stem cells are derived from bone marrow.

Preferred methods include embodiments wherein said mesenchymal stem cells are derived from dermal tissue.

Preferred methods include embodiments wherein said mesenchymal stem cells are derived from umbilical cord.

Preferred methods include embodiments wherein said mesenchymal stem cells are derived from Wharton's Jelly.

Preferred methods include embodiments wherein said mesenchymal stem cells are derived from cord blood.

Preferred methods include embodiments wherein said mesenchymal stem cells are derived from deciduous teeth.

Preferred methods include embodiments wherein said mesenchymal stem cells are differentiated from pluripotent stem cells.

Preferred methods include embodiments wherein said pluripotent stem cells are somatic cell nuclear transplant derived.

Preferred methods include embodiments wherein said pluripotent stem cells are parthenogenesis derived.

Preferred methods include embodiments wherein said pluripotent stem cells are embryonic stem cell derived.

Preferred methods include embodiments wherein said pluripotent stem cells are derived from inducible pluripotent stem cells.

Preferred methods include embodiments wherein said inducible pluripotent stem cells are generated by dedifferentiation of a tissue.

Preferred methods include embodiments wherein said dedifferentiation of said tissue is accomplished by transfection with genes capable of inducing dedifferentiation.

Preferred methods include embodiments wherein said genes capable of inducing dedifferentiation are selected from a group comprising of; a) NANOG; b) OCT4; c) KLF4; d) sox-2; e) c-myc; and f) pim1.

Preferred methods include embodiments wherein said cells are treated with a histone deacetylase inhibitor prior to gene transfection.

Preferred methods include embodiments wherein said histone deacetylase inhibitor is siRNA targeting one or more histone deacetylases.

Preferred methods include embodiments wherein said histone deacetylase inhibitor is microRNA targeting one or more histone deacetylases.

Preferred methods include embodiments wherein said histone deacetylase inhibitor is antisense oligonucleotides targeting one or more histone deacetylases.

Preferred methods include embodiments wherein said histone deacetylase inhibitor is decoy oligonucleotides targeting one or more histone deacetylases.

Preferred methods include embodiments wherein said histone deacetylase inhibitor is hammerhead ribozymes targeting one or more histone deacetylases.

Preferred methods include embodiments wherein said histone deacetylase inhibitor is sulforaphane.

Preferred methods include embodiments wherein said histone deacetylase inhibitor is valproic acid.

Preferred methods include embodiments wherein said histone deacetylase inhibitor is trichostatin A.

Preferred methods include embodiments wherein said histone deacetylase inhibitor is phenylbutyrate.

Preferred methods include embodiments wherein said histone deacetylase inhibitor is vorinostat.

Preferred methods include embodiments wherein said histone deacetylase inhibitor is Romidepsin.

Preferred methods include embodiments wherein said histone deacetylase inhibitor is Panobinostat.

Preferred methods include embodiments wherein said histone deacetylase inhibitor is Belinostat.

Preferred methods include embodiments wherein said mesenchymal stem cells are autologous to the recipient.

Preferred methods include embodiments wherein said mesenchymal stem cells are allogeneic to the recipient.

Preferred methods include embodiments wherein said mesenchymal stem cells are xenogeneic to the recipient.

Preferred methods include embodiments wherein mesenchymal stem cell conditioned media is administered alone and/or in conjunction with mesenchymal stem cells in order to promote tolerogenesis.

Preferred methods include embodiments wherein mesenchymal stem cell conditioned media is administered alone and/or in conjunction with mesenchymal stem cells in order to promote regeneration of the transplanted tissue.

Preferred methods include embodiments wherein regeneration of said transplanted tissue is resistance to apoptosis.

Preferred methods include embodiments wherein regeneration of said transplanted tissue is resistance to inflammation.

Preferred methods include embodiments wherein regeneration of said transplanted tissue is resistance to fibrosis.

Preferred methods include embodiments wherein regeneration of said transplanted tissue is activation of progenitor cells endogenous to said transplanted tissue.

Preferred methods include embodiments wherein said conditioned media is collected from mesenchymal stem cells growing in a liquid media.

Preferred methods include embodiments wherein said liquid media is a serum free media.

Preferred methods include embodiments wherein said liquid media is RPMI.

Preferred methods include embodiments wherein said liquid media is EMEM.

Preferred methods include embodiments wherein said liquid media is alpha MEM.

Preferred methods include embodiments wherein said liquid media is DMEM.

Preferred methods include embodiments wherein said liquid media is Aim-V media.

Preferred methods include embodiments wherein said liquid media is Iscove's media.

Preferred methods include embodiments wherein said conditioned media is generated by culture of mesenchymal stem cells under conditions capable of activating hypoxia inducible factor in said mesenchymal stem cells.

Preferred methods include embodiments wherein said mesenchymal stem cells are grown under hypoxic conditions.

Preferred methods include embodiments wherein said mesenchymal stem cells are grown in the presence of a hypoxia-mimicking compound.

Preferred methods include embodiments wherein said mesenchymal stem cells are activated before being used to generate conditioned media.

Preferred methods include embodiments wherein said activation of mesenchymal stem cells is accomplished by culture with allogenic T cells.

Preferred methods include embodiments wherein said allogeneic T cells are activated before exposure to said mesenchymal stem cells.

Preferred methods include embodiments wherein said T cells are CD3 cells.

Preferred methods include embodiments wherein said T cells are CD4 cells.

Preferred methods include embodiments wherein said T cells are CD8 cells.

Preferred methods include embodiments wherein said T cells are isolated based on expression of ICOS-1.

Preferred methods include embodiments wherein said T cells are isolated based on expression of CD28.

Preferred methods include embodiments wherein said T cells are isolated based on expression of CTLA4.

Preferred methods include embodiments wherein said T cells are isolated based on expression of CD25.

Preferred methods include embodiments wherein said T cells are isolated based on expression of ICAM-1.

Preferred methods include embodiments wherein said T cells are isolated based on expression of LFA3.

Preferred methods include embodiments wherein said T cells are isolated based on expression of CD45.

Preferred methods include embodiments wherein said T cells are isolated based on expression of CD56.

Preferred methods include embodiments wherein said T cells are isolated based on expression of CD57.

Preferred methods include embodiments wherein said T cells are activated by exposure to anti-CD3.

Preferred methods include embodiments wherein said T cells are activated by exposure to anti-CD28.

Preferred methods include embodiments wherein said T cells are activated by exposure to anti-CD3 and anti-CD28.

Preferred methods include embodiments wherein said T cells are activated by exposure to interleukin-2.

Preferred methods include embodiments wherein said T cells are activated by exposure to interleukin-2 and anti-CD3.

Preferred methods include embodiments wherein said T cells are activated by exposure to interleukin-2, anti-CD3 and anti-CD28.

Preferred methods include embodiments wherein said T cells are activated by exposure to a mitogen.

Preferred methods include embodiments wherein said mitogen is a lectin.

Preferred methods include embodiments wherein said lectin is concanavalin A.

Preferred methods include embodiments wherein said lectin is phytohemagglutinin.

Preferred methods include embodiments wherein said lectin is pokeweed mitogen.

Preferred methods include embodiments wherein said conditioned media is generated by culture of mesenchymal stem cells with an activator of a toll like receptor.

Preferred methods include embodiments wherein said toll like receptor agonist is HMGB1.

Preferred methods include embodiments wherein said toll like receptor agonist is flagellin.

Preferred methods include embodiments wherein said toll like receptor agonist is Poly IC.

### DETAILED DESCRIPTION OF THE INVENTION

The invention provides means of utilizing mesenchymal stem cells, modified mesenchymal stem cells, and products derived from said mesenchymal stem cells for induction of “immune tolerance” in the context of transplantation. This term refers to a state of unresponsiveness of the immune system to specific substances or tissues that have the capacity to elicit an immune response while preserving immune responses against other substances or tissues. As used herein, the term “immune response” includes T cell mediated and/or B cell mediated immune responses. Exemplary immune responses include T cell responses, e.g., cytokine production and cellular cytotoxicity, in addition, the term immune response includes immune responses that are indirectly affected by T cell activation, e.g., antibody production (humoral responses) and activation of cytokine responsive cells, e.g., macrophages. Immune cells involved in the immune response include lymphocytes, such as B cells and T cells (CD4+, CD8+, Th1 and Th2 cells); antigen presenting cells (e.g. professional antigen presenting cells such as dendritic cells); natural killer cells; myeloid cells, such as macrophages, eosinophils, mast cells, basophils, and granulocytes.

“Administering” as used herein, refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab).sub.2, as well as single chain antibodies and humanized antibodies. The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. .kappa. and .lamda. light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

The term “auto-antigen” means, in accordance with the present invention, any self-antigen which is mistakenly recognized by the immune system as being foreign. Auto-antigens comprise, but are not limited to, cellular proteins, phosphoproteins, cellular surface proteins, cellular lipids, nucleic acids, glycoproteins, including cell surface receptors.

As used herein, the term “autologous” is meant to refer to any material derived from the same individual to which it is later to be re-introduced into the individual.

“Cytokine,” as used herein, refers to a non-antibody protein that is released by one cell in response to contact with a specific antigen, wherein the cytokine interacts with a second cell to mediate a response in the second cell. A cytokine can be endogenously expressed by a cell or administered to a subject. Cytokines may be released by immune cells, including macrophages, B cells, T cells, and mast cells to propagate an immune response. Cytokines can induce various responses in the recipient cell. Cytokines can include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines can promote an inflammatory response. Examples of homeostatic cytokines include, but are not limited to, IL-2, IL-4, IL-5, IL-7, IL-10, IL-12p40, IL-12p70, IL-15, and interferon (IFN) gamma. Examples of pro-inflammatory cytokines include, but are not limited to, IL-1a, IL-1b, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perforin. Examples of acute phase-proteins include, but are not limited to, C-reactive protein (CRP) and serum amyloid A (SAA).

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” as used herein, means an amount which provides a therapeutic or prophylactic benefit.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “lymphocyte” means T cells, B cells, NK cells, and Lymphokine Activated Killer (LAK) cells. T-lymphocytes possess T-cell receptors, B-lymphocytes, possess B cell receptors and produce antibodies, Tumor Infiltrating Lymphocytes (TIL) are isolated from tumors and possess some degree of reactivity towards the tumor, cytotoxic T lymphocytes (CTL) are lymphocytes of the CD8 lineage usually and possess ability to kill cells through perforin and/or granzymes. CTL isolation means are described in numerous references including U.S. Pat. Nos. 6,805,861 and 6,531,451. Any one lymphocyte produces one type of TCR or antibody. Each TCR or antibody has specificity for one particular epitope, or antigen binding site, on its cognate antigen. Specific TCRs or antibodies are encoded by genes that are formed from the rearrangement of DNA in a lymphocyte stem cell that encodes the constant (“C”), joining (“J”), variable (“V”) regions, and possibly diversity (“D”) regions of the TCR or antibody. Mammals typically possess one-hundred thousand to one-hundred million lymphocytes of different specificities. Upon stimulation of lymphocytes by an antigen, those lymphocytes specific for the antigen undergo clonal amplification. T lymphocytes are formed in the bone marrow, migrate to and mature in the thymus and then enter the peripheral blood and lymphatic circulation. T lymphocytes are subdivided into three distinct types of cells: helper T cells, suppressor T cells, and cytotoxic T cells. T lymphocytes, unlike B lymphocytes, do not produce antibody molecules, but express a heterodimeric cell surface receptor that recognizes peptide fragments of antigenic proteins that are attached to proteins of the major histocompatibility complex (MHC) and expressed on the surfaces of target cells. T lymphocytes include tumor-infiltrating lymphocytes. Cytotoxic T lymphocytes (CTL) are well known in the art and are typically of the CD3+, CD8+, CD4-phenotype. They typically lyse cells that display fragments of foreign antigens associated with class I MHC molecules on their cell surfaces. CTL typically recognize normal cells expressing antigens after infection by viruses or other pathogens; and tumor cells that have undergone transformation and are expressing mutated proteins or are over-expressing normal proteins. Natural Killer (NK) cells are well known in the art. NK cells are a subset of lymphocytes active in the immune system and representing an average 15% of mononuclear cells in human peripheral blood. Among the surface markers used to identify human NK cells is a receptor binding with low affinity to the Fc fragment of IgG antibodies, such as Fc-.gamma. receptor III or CD16 antigen. NK cells have been demonstrated to play an important role in vivo in the defense against tumors, tumor metastases, virus infection, and to regulate normal and malignant hematopoiesis. Lymphokine-activated killer (LAK) cells are well known in the art and are a cytotoxic population of cells which are capable of lysing autologous tumor cells and NK-cell resistant tumor cell lines. Precursors of LAK cells belong to the subpopulation of “null” lymphocytes that bear neither T nor B cell surface markers. In the human these precursor cells are widely found in peripheral blood, lymph nodes, bone marrow and the thoracic duct. Purification of LAK cells, and their generation are described in U.S. Pat. Nos. 5,002,879, 4,849,329 and 4,690,915.

Transplantation tolerance may be induced according to the invention by administration of mesenchymal stem cell populations. In one embodiment said mesenchymal stem cell populations are gene engineered to possess enhanced tolerogenic properties. Such gene engineering may be accomplished by use of standard transfection techniques, or insertion at the genomic level using gene editing. Various genes maybe useful for induction of tolerogenesis. In one embodiment cells of the mesenchymal lineage are transfected with interleukin-10 and/or interleukin-35. Methods of deriving cord tissue mesenchymal stem cells from human umbilical tissue are provided. The cells are capable of self-renewal and expansion in culture, and have the potential to differentiate into cells of other phenotypes. The method comprises (a) obtaining human umbilical tissue; (b) removing substantially all of blood to yield a substantially blood-free umbilical tissue, (c) dissociating the tissue by mechanical or enzymatic treatment, or both, (d) resuspending the tissue in a culture medium, and (e) providing growth conditions which allow for the growth of a human umbilicus-derived cell capable of self-renewal and expansion in culture and having the potential to differentiate into cells of other phenotypes. Tissue can be obtained from any completed pregnancy, term or less than term, whether delivered vaginally, or through other routes, for example surgical Cesarean section. Obtaining tissue from tissue banks is also considered within the scope of the present invention. The tissue is rendered substantially free of blood by any means known in the art. For example, the blood can be physically removed by washing, rinsing, and diluting and the like, before or after bulk blood removal for example by suctioning or draining. Other means of obtaining a tissue substantially free of blood cells might include enzymatic or chemical treatment.

Dissociation of the umbilical tissues can be accomplished by any of the various techniques known in the art, including by mechanical disruption, for example, tissue can be aseptically cut with scissors, or a scalpel, or such tissue can be otherwise minced, blended, ground, or homogenized in any manner that is compatible with recovering intact or viable cells from human tissue. In a presently preferred embodiment, the isolation procedure also utilizes an enzymatic digestion process. Many enzymes are known in the art to be useful for the isolation of individual cells from complex tissue matrices to facilitate growth in culture. As discussed above, a broad range of digestive enzymes for use in cell isolation from tissue is available to the skilled artisan. Ranging from weakly digestive (e.g. deoxyribonucleases and the neutral protease, dispase) to strongly digestive (e.g. papain and trypsin), such enzymes are available commercially. A nonexhaustive list of enzymes compatible herewith includes mucolytic enzyme activities, metalloproteases, neutral proteases, serine proteases (such as trypsin, chymotrypsin, or elastase), and deoxyribonucleases. Presently preferred are enzyme activites selected from metalloproteases, neutral proteases and mucolytic activities. For example, collagenases are known to be useful for isolating various cells from tissues. Deoxyribonucleases can digest single-stranded DNA and can minimize cell-clumping during isolation. Enzymes can be used alone or in combination. Serine protease are preferably used in a sequence following the use of other enzymes as they may degrade the other enzymes being used. The temperature and time of contact with serine proteases must be monitored. Serine proteases may be inhibited with alpha 2 microglobulin in serum and therefore the medium used for digestion is preferably serum-free. EDTA and DNase are commonly used and may improve yields or efficiencies. Preferred methods involve enzymatic treatment with for example collagenase and dispase, or collagenase, dispase, and hyaluronidase, and such methods are provided wherein in certain preferred embodiments, a mixture of collagenase and the neutral protease dispase are used in the dissociating step. More preferred are those methods which employ digestion in the presence of at least one collagenase from Clostridium histolyticum, and either of the protease activities, dispase and thermolysin. Still more preferred are methods employing digestion with both collagenase and dispase enzyme activities. Also preferred are methods which include digestion with a hyaluronidase activity in addition to collagenase and dispase activities. The skilled artisan will appreciate that many such enzyme treatments are known in the art for isolating cells from various tissue sources. For example, the LIBERASE BLENDZYME (Roche) series of enzyme combinations of collagenase and neutral protease are very useful and may be used in the instant methods. Other sources of enzymes are known, and the skilled artisan may also obtain such enzymes directly from their natural sources. The skilled artisan is also well-equipped to assess new, or additional enzymes or enzyme combinations for their utility in isolating the cells of the invention. Preferred enzyme treatments are 0.5, 1, 1.5, or 2 hours long or longer. In other preferred embodiments, the tissue is incubated at 37.degree. C. during the enzyme treatment of the dissociation step. Diluting the digest may also improve yields of cells as cells may be trapped within a viscous digest.

While the use of enzyme activites is presently preferred, it is not required for isolation methods as provided herein. Methods based on mechanical separation alone may be successful in isolating the instant cells from the umbilicus as discussed above.

The cells can be resuspended after the tissue is dissociated into any culture medium as discussed herein above. Cells may be resuspended following a centrifugation step to separate out the cells from tissue or other debris. Resuspension may involve mechanical methods of resuspending, or simply the addition of culture medium to the cells.

In order to properly generate tolerogenic mesenchymal stem cells it is important that the growth conditions allows for a wide range of options as to culture medium, supplements, atmospheric conditions, and relative humidity for the cells. A preferred temperature is 37.degree. C., however the temperature may range from about 35.degree. C. to 39.degree. C. depending on the other culture conditions and desired use of the cells or culture. Presently preferred are methods which provide cells which require no exogenous growth factors, except as are available in the supplemental serum provided with the Growth Medium. Also provided herein are methods of deriving umbilical cells capable of expansion in the absence of particular growth factors. The methods are similar to the method above, however they require that the particular growth factors (for which the cells have no requirement) be absent in the culture medium in which the cells are ultimately resuspended and grown in. In this sense, the method is selective for those cells capable of division in the absence of the particular growth factors. Preferred cells in some embodiments are capable of growth and expansion in chemically-defined growth media with no serum added. In such cases, the cells may require certain growth factors, which can be added to the medium to support and sustain the cells. Presently preferred factors to be added for growth on serum-free media include one or more of FGF, EGF, IGF, and PDGF. In more preferred embodiments, two, three or all four of the factors are add to serum free or chemically defined media. In other embodiments, LIF is added to serum-free medium to support or improve growth of the cells.

Also provided are methods wherein the cells can expand in the presence of from about 5% to about 20% oxygen in their atmosphere. Methods to obtain cells that require L-valine require that cells be cultured in the presence of L-valine. After a cell is obtained, its need for L-valine can be tested and confirmed by growing on D-valine containing medium that lacks the L-isomer.

Methods are provided wherein the cells can undergo at least 25, 30, 35, or 40 doublings prior to reaching a senescent state. Methods for deriving cells capable of doubling to reach 10.sup.14 cells or more are provided. Preferred are those methods which derive cells that can double sufficiently to produce at least about 10.sup.14, 10.sup.15, 10.sup.16, or 10.sup.17 or more cells when seeded at from about 10.sup.3 to about 10.sup.6 cells/cm.sup.2 in culture. Preferably these cell numbers are produced within 80, 70, or 60 days or less. In one embodiment, cord tissue mesenchymal stem cells are isolated and expanded, and possess one or more markers selected from a group comprising of CD10, CD13, CD44, CD73, CD90, CD141, PDGFr-alpha, or HLA-A,B,C. In addition, the cells do not produce one or more of CD31, CD34, CD45, CD117, CD141, or HLA-DR,DP, DQ.

In one embodiment, bone marrow MSC lots are generated, means of generating BM MSC are known in the literature and examples are incorporated by reference.

In one embodiment BM-MSC are generated as follows
1. 500 mL Isolation Buffer is prepared (PBS+2% FBS+2 mM EDTA) using sterile components or filtering Isolation Buffer through a 0.2 micron filter. Once made, the Isolation Buffer was stored at 2-8. degree. C.
2. The total number of nucleated cells in the BM sample is counted by taking 10.mu.L BM and diluting it 1/50-1/100 with 3% Acetic Acid with Methylene Blue (STEMCELL Catalog #07060). Cells are counted using a hemacytometer.
3. 50 mL Isolation Buffer is warmed to room temperature for 20 minutes prior to use and bone marrow was diluted 5/14 final dilution with room temperature Isolation Buffer (e.g. 25 mL BM was diluted with 45 mL Isolation Buffer for a total volume of 70 mL).
4. In three 50 ml conical tubes (BD Catalog #352070), 17 mL Ficoll-Paque™ PLUS (Catalog #07907/07957) is pipetted into each tube. About 23 mL of the diluted BM from step 3 was carefully layered on top of the Ficoll-Paque™ PLUS in each tube.
5. The tubes are centrifuged at room temperature (15-25.degree. C.) for 30 minutes at 300.times.g in a bench top centrifuge with the brake off.
6. The upper plasma layer is removed and discarded without disturbing the plasma: Ficoll-Paque™ PLUS interface. The mononuclear cells located at the interface layer are carefully removed and placed in a new 50 ml conical tube. Mononuclear cells are resuspended with 40 ml cold (2-8. degree. C.) Isolation Buffer and mixed gently by pipetting.
7. Cells were centrifuged at 300.times.g for 10 minutes at room temperature in a bench top centrifuge with the brake on. The supernatant is removed and the cell pellet resuspended in 1-2 mL cold Isolation Buffer.
8. Cells were diluted 1/50 in 3% Acetic Acid with Methylene Blue and the total number of nucleated cells counted using a hemacytometer.
9. Cells are diluted in Complete Human MesenCult®-Proliferation medium (STEMCELL catalog #05411) at a final concentration of 1.times.10.sup.6 cells/mL.
10. BM-derived cells were ready for expansion and CFU-F assays in the presence of GW2580, which can then be used for specific applications.

In one embodiment, MSC are generated according to protocols previously utilized for treatment of patients utilizing bone marrow derived MSC. Specifically, bone marrow is aspirated (10-30 ml) under local anesthesia (with or without sedation) from the posterior iliac crest, collected into sodium heparin containing tubes and transferred to a Good Manufacturing Practices (GMP) clean room. Bone marrow cells are washed with a washing solution such as Dulbecco's phosphate-buffered saline (DPBS), RPMI, or PBS supplemented with autologous patient plasma and layered on to 25 ml of Percoll (1.073 g/ml) at a concentration of approximately 1-2′ 107 cells/ml. Subsequently the cells are centrifuged at 900 g for approximately 30 min or a time period sufficient to achieve separation of mononuclear cells from debris and erythrocytes. Said cells are then washed with PBS and plated at a density of approximately 1′ 106 cells per ml in 175 cm2 tissue culture flasks in DMEM with 10% FCS with flasks subsequently being loaded with a minimum of 30 million bone marrow mononuclear cells. The MSCs are allowed to adhere for 72 h followed by media changes every 3-4 days. Adherent cells are removed with 0.05% trypsin-EDTA and replated at a density of 1′ 106 per 175 cm2. Said bone marrow MSC may be administered intravenously, or in a preferred embodiment, intrathecally in a patient suffering radiation associated neurodegenerative manifestations. Although doses may be determined by one of skill in the art, and are dependent on various patient characteristics, intravenous administration may be performed at concentrations ranging from 1-10 million MSC per kilogram, with a preferred dose of approximately 2-5 million cells per kilogram.

In order to determine the quality of MSC cultures, flow cytometry is performed on all cultures for surface expression of SH-2, SH-3, SH-4 MSC markers and lack of contaminating CD14- and CD-45 positive cells. Cells were detached with 0.05% trypsin-EDTA, washed with DPBS+2% bovine albumin, fixed in 1% paraformaldehyde, blocked in 10% serum, incubated separately with primary SH-2, SH-3 and SH-4 antibodies followed by PE-conjugated anti-mouse IgG (H+L) antibody. Confluent MSC in 175 cm2 flasks are washed with Tyrode's salt solution, incubated with medium 199 (M199) for 60 min, and detached with 0.05% trypsin-EDTA (Gibco). Cells from 10 flasks were detached at a time and MSCs were resuspended in 40 ml of M199+1% human serum albumin (HSA; American Red Cross, Washington DC, USA). MSCs harvested from each 10-flask set were stored for up to 4 h at 4° C. and combined at the end of the harvest. A total of 2-10′ 106 MSC/kg were resuspended in M199+1% HSA and centrifuged at 460 g for 10 min at 20° C. Cell pellets were resuspended in fresh M199+1% HSA media and centrifuged at 460 g for 10 min at 20° C. for three additional times. Total harvest time was 2-4 h based on MSC yield per flask and the target dose. Harvested MSC were cryopreserved in Cryocyte (Baxter, Deerfield, IL, USA) freezing bags using a rate controlled freezer at a final concentration of 10% DMSO (Research Industries, Salt Lake City, UT, USA) and 5% HSA. On the day of infusion cryopreserved units were thawed at the bedside in a 37° C. water bath and transferred into 60 ml syringes within 5 min and infused intravenously into patients over 10-15 min. Patients are premedicated with 325-650 mg acetaminophen and 12.5-25 mg of diphenhydramine orally. Blood pressure, pulse, respiratory rate, temperature and oxygen saturation are monitored at the time of infusion and every 15 min thereafter for 3 h followed by every 2 h for 6 h.

Within the context of the invention, exosomes and microparticles may be used interchangeably. Exosomes from MSC may be generated from a mesenchymal stem cell conditioned medium (MSC-CM). Said exosomes are used in the context of the invention to reprogram immunocytes for tolerance induction ex vivo or in vivo. Said particle may be isolated for example by being separated from non-associated components based on any property of the particle. For example, the particle may be isolated based on molecular weight, size, shape, composition or biological activity. The conditioned medium may be filtered or concentrated or both during, prior to or subsequent to separation. For example, it may be filtered through a membrane, for example one with a size or molecular weight cut-off. It may be subject to tangential force filtration or ultrafiltration.

With regard to a donor tissue, cell, graft or solid organ transplant in a recipient patient, it is believed that the method according to the invention may be effective in preventing acute rejection of such transplant in the recipient and/or for long-term maintenance therapy to prevent rejection of such transplant in the recipient (e.g., inhibiting rejection of insulin-producing islet cell transplant from a donor in the patient recipient suffering from diabetes). Thus, the method of the invention is useful for preventing Host-Versus-Graft-Disease (HVGD) and Graft-Versus-Host-Disease (GVHD). Typically, the method of the present invention is applied to the patient before and/or after transplantation. As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By a “therapeutically effective amount” is meant a sufficient amount of cells generated with the present invention for the treatment of the disease at a reasonable benefit/risk ratio applicable to any medical treatment. It will be understood that the total usage of these cells will be decided by the attending physicians within the scope of sound medical judgment. The specific therapeutically effective dose level for any particular patient will depend upon a variety of factors including the age, body weight, general health, sex and diet of the patient; the time of administration, route of administration, and survival rate of the cells employed; the duration of the treatment; drugs used in combination or coincidental with the administered cells; and like factors well known in the medical arts. For example, it is well known within the skill of the art to start doses of cells at levels lower than those required to achieve the desired therapeutic effect and to gradually increase the dosage until the desired effect is achieved.

The invention provides novel stem cell types, methods of manufacture, and therapeutic uses. Provided are means of deriving stem cells possessing regenerative, immune modulatory, anti-inflammatory, and angiogenic/neurogenic activity from umbilical cord tissue such as Wharton's Jelly. In some embodiments manipulation of stem cell “potency” is disclosed through hypoxic manipulation, growth on non-xenogeneic conditions, as well as addition of epigenetic modulators.

Mesenchymal stem cells may be encapsulated by membranes, as well as capsules, prior to implantation. It is contemplated that any of the many methods of cell encapsulation available may be employed. In some embodiments, cells are individually encapsulated. In some embodiments, many cells are encapsulated within the same membrane. In embodiments in which the cells are to be removed following implantation, a relatively large size structure encapsulating many cells, such as within a single membrane, may provide a convenient means for retrieval. A wide variety of materials may be used in various embodiments for microencapsulation of Reprogrammed immune cells. Such materials include, for example, polymer capsules, alginate-poly-L-lysine-alginate microcapsules, barium poly-L-lysine alginate capsules, barium alginate capsules, polyacrylonitrile/polyvinylchloride (PAN/PVC) hollow fibers, and polyethersulfone (PES) hollow fibers. Techniques for microencapsulation of cells that may be used for administration of Reprogrammed immune cells are known to those of skill in the art and are described, for example, in Chang, P., et al., 1999; Matthew, H. W., et al., 1991; Yanagi, K., et al., 1989; Cai Z. H., et al., 1988; Chang, T. M., 1992 and in U.S. Pat. No. 5,639,275 (which, for example, describes a biocompatible capsule for long-term maintenance of cells that stably express biologically active molecules. Additional methods of encapsulation are in European Patent Publication No. 301,777 and U.S. Pat. Nos. 4,353,888; 4,744,933; 4,749,620; 4,814,274; 5,084,350; 5,089,272; 5,578,442; 5,639,275; and 5,676,943. All of the foregoing are incorporated herein by reference in parts pertinent to encapsulation of Reprogrammed immune cells. The cells of the invention are cultured under hypoxia, in one embodiment, cultured in order to induce and/or augment expression of chemokine receptors. One such receptor is CXCR-4. The population of cells, including population of umbilical cord mesenchymal cells, may be enriched for CXCR-4, such as (or such as about) 50%, 55%, 60%, 65%, 70%, 75%, 80%, 85%, 90%, 95%, 96%, 97%, 98%, 99%, or more of the population expressing CXCR-4, CD31, CD34, or any combination thereof. In addition or alternatively, <1%, <2%, <3%, <4%, <5%, <6%, <7%, <8%, <9%, or <10% of the population of cells may express CD14 and/or CD45. The umbilical cord cells of the invention may further possess markers selected from the group consisting of STRO-1, CD105, CD54, CD56, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1, and a combination thereof. In some embodiments said placental cells of the invention are admixed with endothelial cells. Said endothelial cells may express one or more markers selected from the group consisting of: a) extracellular vimentin; b) CD133; c) c-kit; d) VEGF receptor; e) activated protein C receptor; and f) a combination thereof. In some embodiments, the population of endothelial cells comprises endothelial progenitor cells.

The population of cells may be allogeneic, autologous, or xenogenic to an individual, including an individual being administered the population of cells. In some embodiments, the population of cells are matched by mixed lymphocyte reaction matching.

In some embodiments, the population of cells is derived from tissue selected from the group consisting of the placental body, placenta, umbilical cord tissue, peripheral blood, hair follicle, cord blood, Wharton's Jelly, menstrual blood, endometrium, skin, omentum, amniotic fluid, and a combination thereof. In some embodiments, the population of cells, the population of umbilical mesenchymal stem cells, or the population of endothelial cells comprises human umbilical cord derived adherent cells. The human umbilical cord derived adherent cells may express a cytokines selected from the group consisting of) FGF-1; b) FGF-2; c) HGF; d) interleukin-1 receptor antagonist; and e) a combination thereof. In some embodiments, the population of cells, the population of umbilical cord cells express arginase, indoleamine 2,3 deoxygenase, interleukin-10, and/or interleukin 35. In some embodiments, the population of cells, the population of umbilical cord cells, or the population of endothelial cells express hTERT and Oct-4 but does not express a STRO-1 marker.

In one embodiment the invention provides the utilization of mesenchymal stem cells for generation of tolerance promoting cells such as tolerogenic dendritic cells and/or T regulatory cells. The stimulation of these therapeutic cell populations may be further performed by administration of mesenchymal stem cells together with “tolerogenic adjuvants” such as anti-CD3 antibodies, anti-CD45RB antibodies or low dose interleukin-2. “Mesenchymal stem cell” or “MSC” in some embodiments refers to cells that are (1) adherent to plastic, (2) express CD73, CD90, and CD105 antigens, while being CD14, CD34, CD45, and HLA-DR negative, and (3) possess ability to differentiate to osteogenic, chondrogenic and adipogenic lineage. Other cells possessing mesenchymal-like properties are included within the definition of “mesenchymal stem cell”, with the condition that said cells possess at least one of the following: a) regenerative activity; b) production of growth factors; c) ability to induce a healing response, either directly, or through elicitation of endogenous host repair mechanisms. As used herein, “mesenchymal stromal cell” or ore mesenchymal stem cell can be used interchangeably. Said MSC can be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. In some definitions of “MSC”, said cells include cells that are CD34 positive upon initial isolation from tissue but are similar to cells described about phenotypically and functionally. As used herein, “MSC” may includes cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. Furthermore, as used herein, in some contexts, “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, Ixmyelocel-T, MSC-NTF, NurOwn™, Stemedyne™-MSC, Stempeucel®, StempeuceICLI, StempeuceIOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs). the population of umbilical cord cells has an ability to undergo cell division in less than 36 hours in a growth medium. In some embodiments, the population of cells, the population of umbilical cord cells has an ability to proliferate at a rate of 0.9-1.2 doublings per 36 hours in growth media. In some embodiments, the population of cells, the population of umbilical cord cells has an ability to proliferate at a rate of 0.9, 1.0, 1.1, or 1.2 doublings per 36 hours in growth media. The population of cells, population of umbilical cord cells may produce exosomes capable of inducing more than 50% proliferation when the exosomes are cultured with human umbilical cord endothelial cells. The induction of proliferation may occur when the exosomes are cultured with the human umbilical cord endothelial cells at a concentration of 10, 20, 30, 40, 50, 60, 70, 80, 90, 100, 110, 120, 130, 140, 150, 160, 170, 180, 190, 200, or more exosomes per cell.

In some embodiments, a population of cells, including a population of umbilical cells alone, are administered to an individual, including an individual having and acute or chronic pathology, wherein the population of cells may be administered via any suitable route, including as non-limiting examples, intramuscularly and/or intravenously.

In some embodiments, a population of umbilical cord cells is optionally obtained, the population is then optionally contacted via culturing with a population of progenitor for T regulatory cells, wherein the culturing conditions allow for the generation of T regulatory cells, then the generated T regulatory cells are administered to an individual.

1. A method of promoting immunological tolerance to a transplanted tissue comprising the steps of: a) identifying immunological factors in a potential transplant recipient; b) administering a mesenchymal stem cell population prior to transplantation of said tissue in a manner to alter recipient immune system; c) assessing recipient immune system modifications induced by said mesenchymal stem cell population; d) performing said transplant; and e) tailoring future administration of said mesenchymal stem cells based on alterations of recipient immune response post-transplant.

2. The method of claim 1, wherein said transplanted tissue is an organ.

3. The method of claim 1, wherein said transplanted tissue is one or more cellular populations.

4. The method of claim 1, wherein said immunological tolerance is a state in which the recipient immune system is actively suppressing immune responses against graft associated antigens.

5. The method of claim 4, wherein said suppression of anti-graft immunity is mediated by tolerogenic dendritic cells.

6. The method of claim 5, wherein said tolerogenic dendritic cells express PD-L1.

7. The method of claim 5, wherein said tolerogenic dendritic cells increase exosome production of activated T cells.

8. The method of claim 7, wherein said exosomes express tetraspanin.

9. The method of claim 7, wherein said exosomes express membrane bound TGF-beta.

10. The method of claim 7, wherein said exosomes express membrane bound IL-10.

11. The method of claim 7, wherein said exosomes express membrane bound IL-35.

12. The method of claim 1, wherein said immunological tolerance is associated with generation of graft protective T regulatory cells.

13. The method of claim 12, wherein said graft protective T regulatory cells are capable of suppressing dendritic cell activation.

14. The method of claim 1, wherein said recipient immunological factor is donor reactive antibodies.

15. The method of claim 1, wherein said recipient immunological factor is donor reactive T cells.

16. The method of claim 1, wherein mesenchymal stem cell conditioned media is administered alone and/or in conjunction with mesenchymal stem cells in order to promote regeneration of the transplanted tissue.

17. The method of claim 16, wherein regeneration of said transplanted tissue is resistance to inflammation.

18. The method of claim 16, wherein regeneration of said transplanted tissue is resistance to fibrosis.

19. The method of claim 16, wherein said conditioned media is generated by culture of mesenchymal stem cells under conditions capable of activating hypoxia inducible factor in said mesenchymal stem cells.

20. The method of claim 19, wherein said mesenchymal stem cells are grown under hypoxic conditions.
